(19) United States
(12) Patent Application Publication
(10) Pub. No.: US 2025/0127972 A1
(43) Pub. Date: Apr. 24, 2025

(54) Implantable Peritoneal Dialysis Catheter with Port IPD Catheter

(71) Applicant: John Allmond, Beaumont, CA (US)

(72) Inventor: John Allmond, Beaumont, CA (US)

(21) Appl. No.: 18/492,797

(22) Filed: Oct. 24, 2023

(51) Int. Cl. A61M 1/28; A61M 25/00; A61M 25/01

(52) U.S. Cl. CPC A61M 1/285; A61M 25/0068; A61M 25/0074; A61M 25/0194; A61M 2025/0197

(57) ABSTRACT

Peritoneal dialysis is delivered via implementation of the total implantable system disclosed. The system is composed of several components. The extended surface area port, equipped with an internal support pillar, is implanted beneath the subcutaneous tissue. The catheter portion is tunneled under the subcutaneous tissue until it reaches the abdomen where it enters the peritoneal cavity. The distal/terminating end of the catheter resides in the peritoneal cavity and is designed with an inner perforated distal catheter, circumferentially enclosed by an outer perforated sheath, creating an anti-occlusion technology system. The distal end of the sheath is designed with an anti-clog tip. Port and system access is achieved via hypodermic needle. Dialysis solution is infused and evacuated via the same modalities.

### FIELD OF THE INVENTION

The declared invention is associated with catheters. This current invention is relating to a totally implantable catheter that utilizes an access port. This catheter will be capable of delivering dialysis solution into the peritoneal cavity for dialysis. This said invention identifies with systems procedures and techniques that implant and utilizes catheters.

### BACKGROUND

At present, there are over half a million people receiving renal dialysis in the US alone. That number rises to over 2 million worldwide. Hemodialysis is a very arduous taxing procedure subjecting patients to imminent morbidity and possible mortality. In addition, according to the Centers for Medicare and Medicaid Services, taxpayers spend more than 50 billion dollars per year providing hemodialysis. That equals nearly one hundred thousand dollars per year per patient.

Peritoneal dialysis (also known as PD) is the alternative to hemodialysis, is less taxing on the body, more convenient, costing 75% less than hemodialysis and it mimics the body's natural filtration process. Its present delivery presents some challenges. Because PD utilizes a catheter to deliver the treatment inside of the abdominal cavity, the catheter permanently remains inserted and extended for microorganisms to enter thus creating a high risk for infections. In addition, the catheter occasionally becomes clogged by fatty tissue in the abdomen called the omentum.

This implantable peritoneal dialysis catheter will virtually eliminate peritonitis caused by the already existing peritoneal dialysis catheters. The dialysis catheters that are already in use are implanted into the perineum and tunnel under the subcutaneous tissue and the end terminates outside of the body. This technique leaves an opening to the outside of the body from the peritoneal cavity. This opening serves as a conduit for the transmission of microorganisms from outside of the body and to the abdomen thus creating a high incident of infection. This device that is presented will be implanted into the peritoneal cavity. The distal end of the catheter will terminate under the subcutaneous tissue. The distal or terminal end will connect to dialysis solution will be injected and to the port by way of the hypodermic needle eliminating a constant opening that will allow microorganisms to enter the peritoneal cavity through plunging of the catheter and the wick effect. Another problem that exists with the conventional peritoneal dialysis catheters is blockage or obstruction on the proximal end caused by the omentum in the abdomen. This invention is designed to prevent occlusion by the omentum by having an inner and outer sheath and as well as perforations on the inner sheath.

### SUMMARY

This said invention provides a novel method for the utilization of catheters for peritoneal dialysis. Traditionally, peritoneal dialysis catheters have implemented the dialysis process by utilizing catheter designs that terminate outside of the body. The presented catheter is designed to be totally implanted in the body. This design will incorporate an access port that will be implanted under the subcutaneous tissue. The port will be accessed via a hypodermic needle that will penetrate the skin through the subcutaneous tissue until it accesses the port. Dialysis solution will be infused in and evacuated out of the device. Said device will render to the patient an increased quantity of life.

### BRIEF DESCRIPTION OF DRAWINGS

FIG. 1 is a plan view according to the precepts and preeminence of the present invention.

FIG. 2 is an exploded view FIG. 1 with a detailed identification of FIG. 1 components.

FIG. 3 is a schematic diagram with three separate perspectives of FIG. 1 and two separate perspectives of the port component of FIG. 1.

FIG. 4A is an enlarged view of the external sheath component of FIG. 1

FIG. 4B is an intraluminal view of FIG. 4

FIG. 5 is an illustrated representation of FIG. 1 regarding its positioning as it relates to the human body after implantation.

FIG. 6 is an illustrated representation of FIG. 1 regarding its positioning as it relates to the profile or trans-lateral view of the human body after implantation, giving emphasis to port positioning.

FIG. 7 is an illustrated representation of FIG. 1 regarding its positioning as it relates to the anterior left oblique view of the human body after its implantation, giving emphasis to the distal catheter and anti-occlusion component positioning.

FIG. 8 is an illustrated representation of FIG. 1 regarding its positioning as it relates to the profile or trans-lateral view of the human body after its implantation into the peritoneal cavity, giving emphasis to the distal catheter and anti-occlusion component positioning.

### DETAILED DESCRIPTION OF DRAWINGS

The present invention can be manufactured in a variety of configurations, it is the currently presented manifestation (designs and descriptions) in this declaration that is prevalent. This manifestation is the exemplification of the general scientific theorems and the applications of the invention to include documentation and does not limit the broad aspects of the invention only to the provided artwork and documentation for the manifestation.

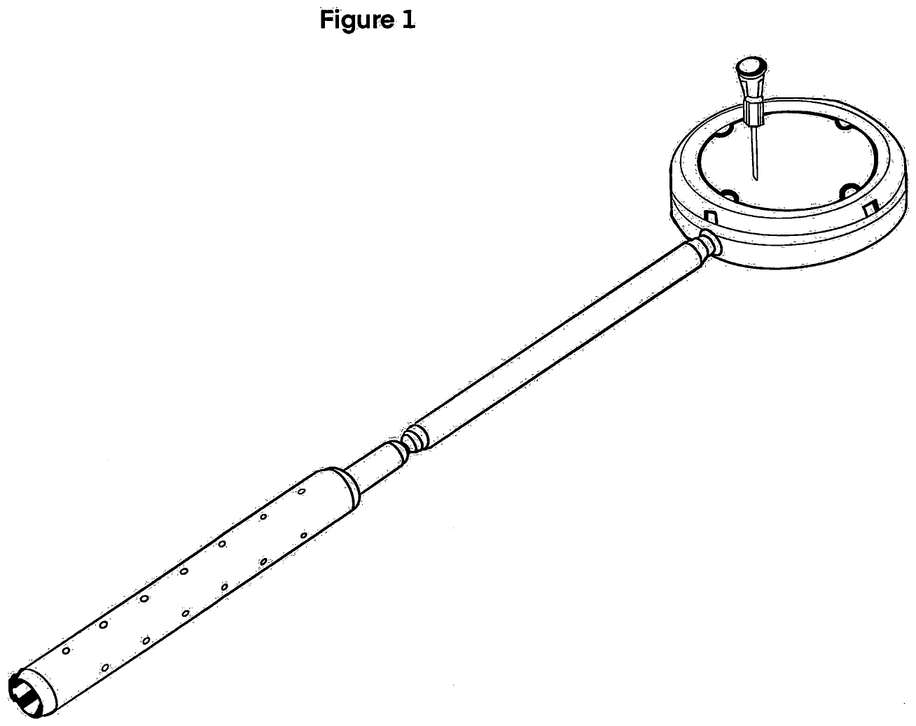

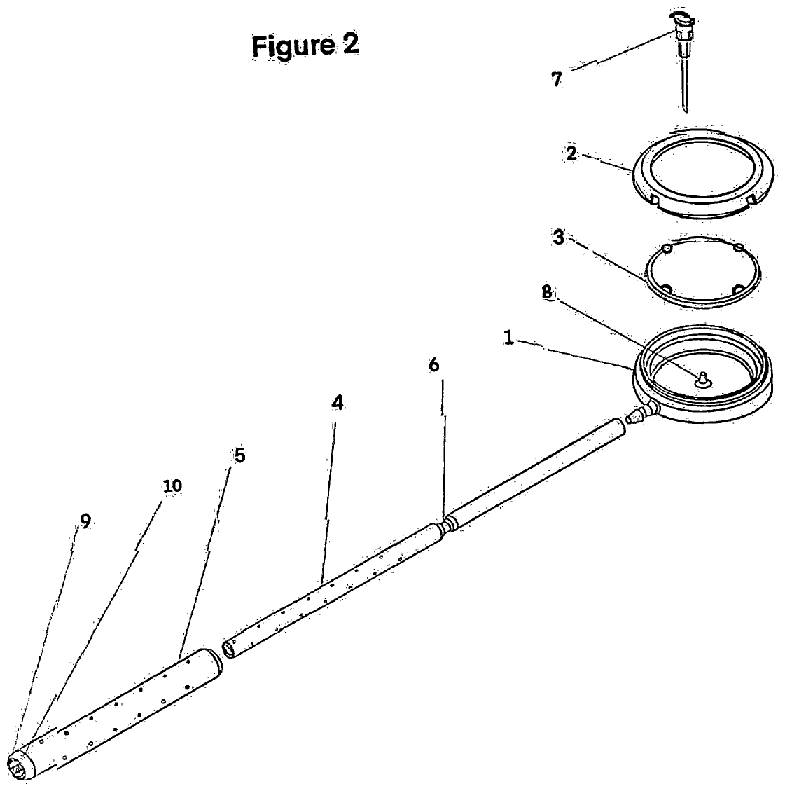

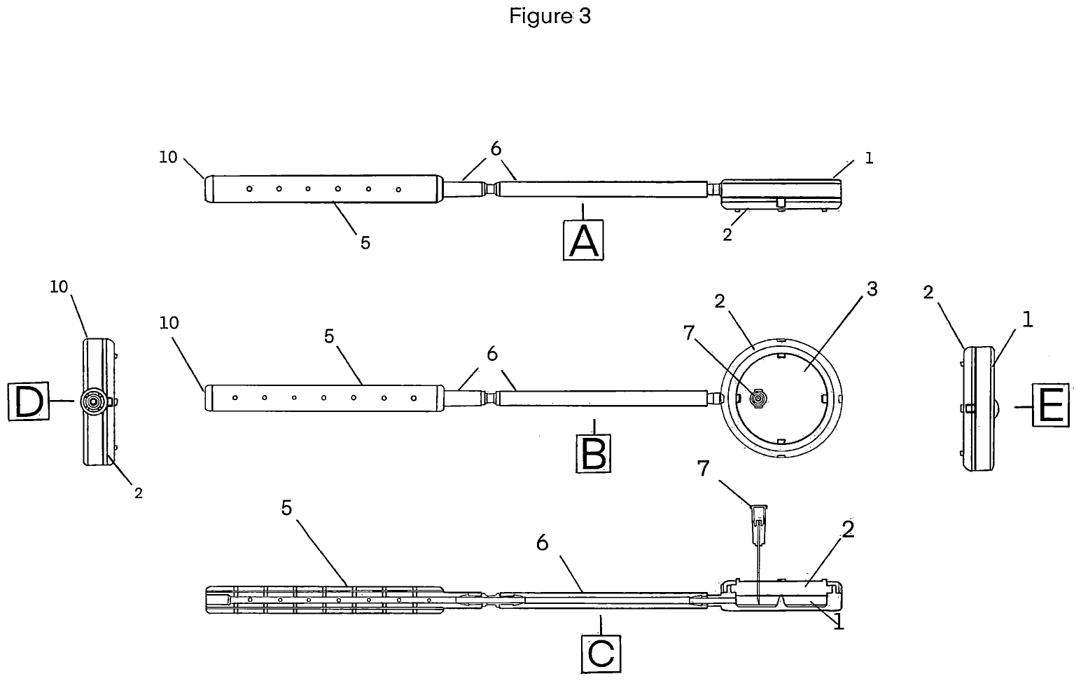

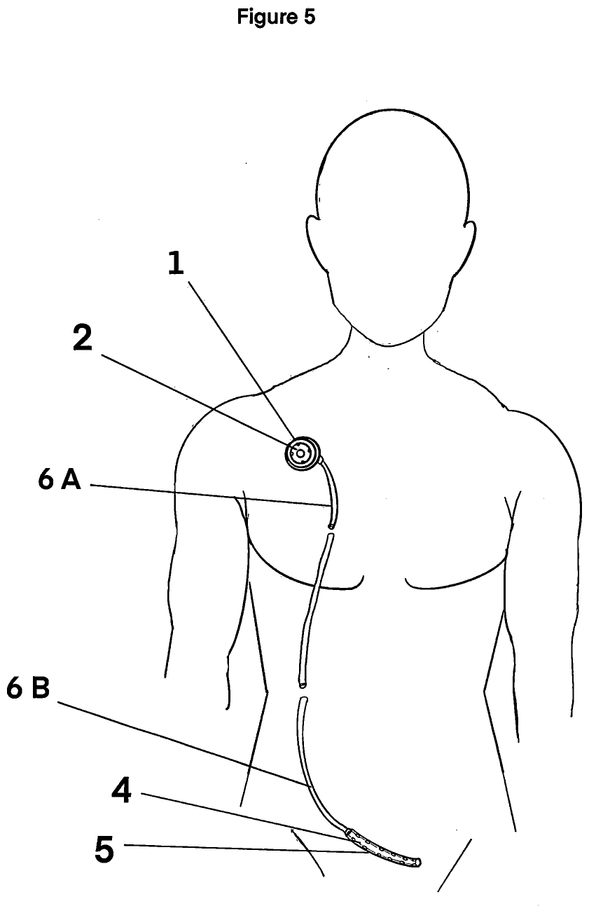

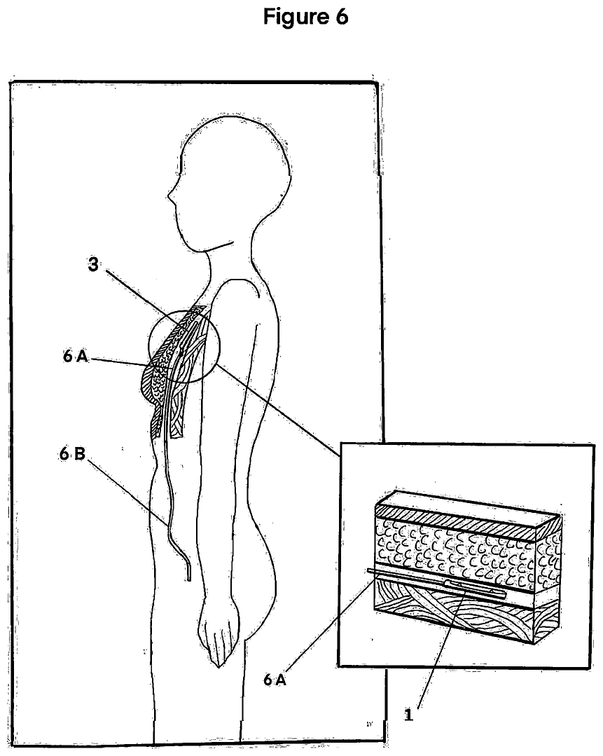

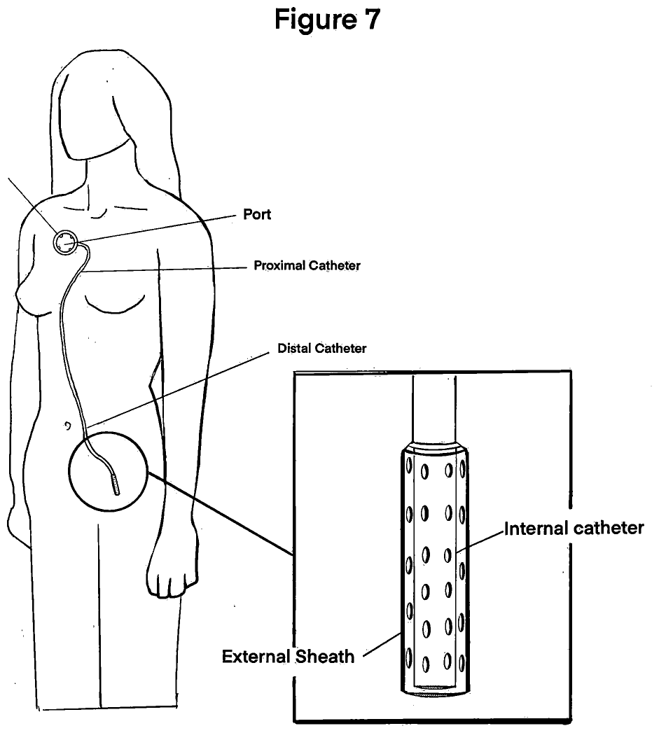

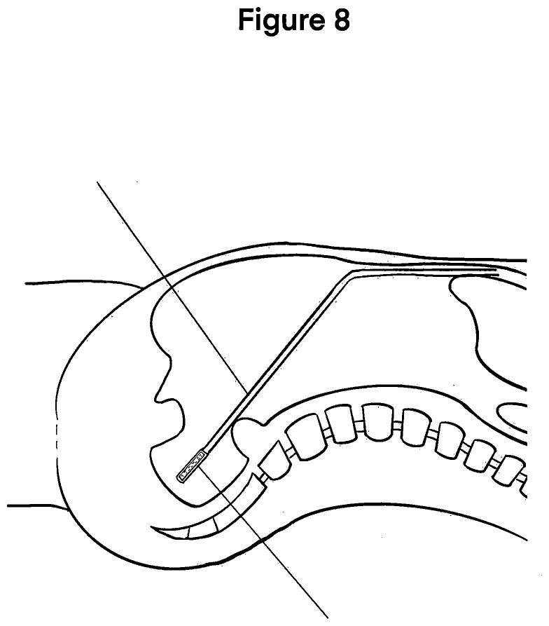

A novel catheter in accordance with the application is exemplified in its totality via this present work in FIG. 1. FIG. 2 is exploded to give perspective to FIG. 1 for comprehension of practical application. FIG. 2 will also shed light as to the functionality of the invention together with a descriptive analysis. The catheter is fully implanted surgically, as exhibited in FIG. 5. The catheter can be implanted through a small incision via laparoscopic procedure, or it can be implanted by way of mini laparotomy surgery, which is dependent on a larger incision for insertion. The catheter can be adjusted to accommodate the patients' size and anatomical differences. With either technique, access to the peritoneum is achieved by way of a peri-umbilical puncture or incision. A stylet is inserted down the center Lumen of the catheter to give it firmness. The distal portion of the catheter is illustrated in FIG. 3-A, 3-B C-6, FIG. 5-6B, FIG. 6-6B, FIG. 7-6B, and FIG. 8-6B is guided into the abdomen at the pelvic region and the stylet is then removed. A surgical instrument, a tunneler is utilized to create a tunnel beneath the subcutaneous tissue terminating at the site of the surgeon's choice. In these illustrations, the terminal point would be the patient's right upper thoracic region near the clavicle. A small incision is made just under the destination site for the port as Illustrated in FIGS. 5 1 and 5-2. Also, this applies to FIG. 6-3. The proximal catheter Illustrated in FIG. 6-6A is fed through the tunneler to the upper incision site at the upper thoracic region. The tunneler is removed, exposing the proximal end of the catheter as observed in 6-6a. A pocket is then created under the subcutaneous tissue by the surgeon. This pocket will house or encapsulate the port as seen and FIG. 3-D, 3-E. The proximal end of the catheter seen in FIGS. 5-A and 6-A is then connected to the port, FIG. 3-D, 3-E. The port is then placed and secured within the prepared pocket under the subcutaneous tissue after a patency test with heparinized saline. The surgical incision is then closed, and the device is left until wound healing until wound healing.

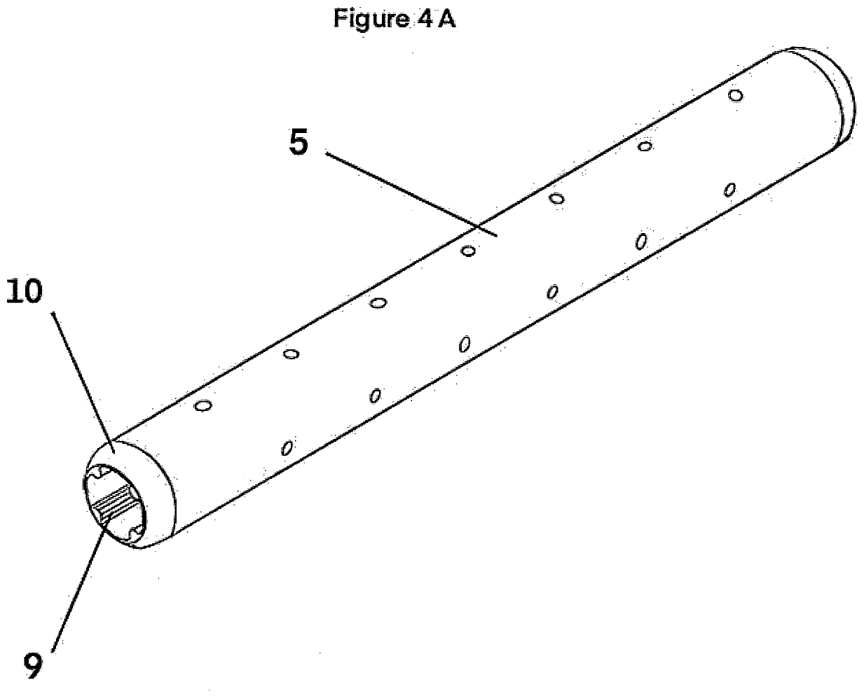

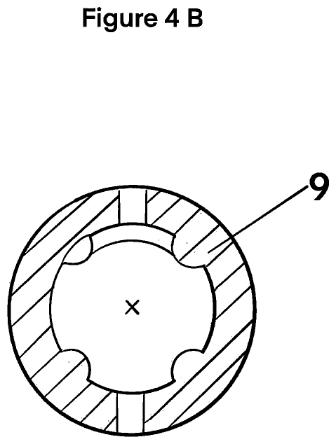

After the predetermined healing period has taken place, the peritoneal dialysis procedure can begin. The peritoneal dialysate fluid tubing will terminate at the distal portion by being connected to a hypodermic needle, which is illustrated in FIGS. 2-7 and FIG. 3-B, C 7. The dialysis solution will then flow from its source into the port, FIG. 3-B, 3-E. The fluid will flow through the needle after the needle has been inserted into a silicone diaphragm FIG. 3-2. The dialysis solution will flow from the needle FIGS. 3-7 and a holding chamber FIG. 3-1 and FIG. 2-1. The holding chamber will contain a support pillar that will add support and prevent collapsing of the diaphragm FIG. 2-3 FIG. 3-2. The dialysis solution will then flow down the catheter from proximal to distal FIG. 5-6A and FIG. 5-6B. The dialysis solution will then exit the distal terminal portion of the catheter in FIGS. 5-4 and 5-5 entering the peritoneal cavity. After dialysis solution has completed its dwell time in the peritoneal cavity, vacuum pressure will be applied to the peritoneal cavity via a needle FIG. 2-7, FIG. 3-B7 FIG. 3-C7 This will create a negative pressure in the abdominal cavity, thus evacuating the spent dialysis solution from the peritoneal cavity. The dialysate solution will travel back through the distal portion of the catheter FIG. 3-5. The dialysate solution will flow through the external sheath of the distal catheter FIG. 5-5. FIG. 8-5, FIG. 7-5. The outer sheet of the distal catheter FIG. 4A-5 will act as a barrier between the internal organs and the inner distal catheter FIG. 2-4 and FIG. 5-4. The outer cannula FIG. 4A will be separated by ribs FIG. 4B to prevent collapse under vacuum pressure. The spent dialysate solution will flow back up and exit the catheter in the opposite direction in which it entered.

| U.S. PATENT DOCUMENTS | REFERENNCES |
|---|---|
| 5,334,139 A 8/1994 | |
| Jeppsson et al. | |
| 5,399,156 A 3/1995 Lindsay | |
| 2,249,473 A 7/1941 Jackson | 5,472,720 A 12/1995 Rakhimov et al. |
| 3,227,877 A 1/1966 Dreyfus | 5,540,265 A 7/1996 Polaschegg et al. |
| 3,391,951 A 7/1968 Miller | 5,542,913 A 8/1996 Lindsay |
| 3,413,097 A 11/1968 Jungner | 5,572,992 A 11/1996 |
| | Kankkunen et al. |
| 3,626,938 A 12/1971 Versaci | 5,583,948 A 12/1996 Shibayama |
| 3,709,222 A 1/1973 Devries | 5,612,001 A 3/1997 Matschke |
| 3,780,736 A 12/1973 Chen | 5,647,984 A 7/1997 Hovland et al. |
| 3,814,680 A 6/1974 Wood | 5,707,911 A 1/1998 Rakhimov et al. |
| 3,840,011 A 10/1974 Wright | 5,714,119 A 2/1998 Kawagoe et al. |
| 3,916,950 A 11/1975 | 5,733,457 A 3/1998 Hovland et al. |
| Mongerson et al. | |
| 3,926,556 A 12/1975 Boucher | 5,792,419 A 8/1998 Williamson et al. |
| 3,955,922 A 5/1976 Moulthrop | 5,843,379 A 12/1998 Kristensen |
| 3,957,082 A 5/1976 Fuson et al. | 5,900,211 A 5/1999 Dunn eta!. |
| 3,986,508 A 10/1976 Barrington | 5,925,014 A 7/1999 Teeple Jr. |
| 3,994,686 A 11/1976 Rauser et al. | 5,948,247 A 9/1999 Gillerfalk et al. |
| 4,056,116 A 11/1977 Carter et al. | 6,013,918 A 1/2000 Bushnell et al. |
| 4,063,890 A 12/1977 Baron | 6,146,600 A 11/2000 Williamson |
| 4,069,153 A 1/1978 Gunther | 6,171,561 B1 1/2001 |
| | Williamson et al. |
| 4,080,965 A 3/1978 Phillips | 6,228,332 B1 5/2001 Dunn eta!. |
| 4,096,859 A 6/1978 Agarwal et al. | 6,234,538 B1 5/2001 Lauer |
| 4,121,107 A 10/1978 Bachmann | 6,245,570 B1 6/2001 Grimm et al. |
| 4,141,686 A 2/1979 Lewis | 6,293,921 B1 9/2001 Shinmoto et al. |
| 4,169,474 A 10/1979 Wagner | 6,440,095 B1 8/2002 Utterberg |
| 4,173,234 A 11/1979 Thomas | 6,443,147 B1 9/2002 Matter |
| 4,196,730 A 4/1980 Wilson | 6,461,568 B1 10/2002 Eckhardt |
| 4,201,917 A 5/1980 Graentzel | 6,468,424 B1 10/2002 Donig et al. |
| 4,219,055 A 8/1980 Wright | 6,565,525 B1 5/2003 Burbank et al. |
| 4,219,221 A 8/1980 Webb | 6,565,803 B1 5/2003 Bolton et al. |
| 4,239,041 A 12/1980 | 6,696,023 B2 2/2004 Grimm et al. |
| Popovich et al. | |
| 4,242,310 A 12/1980 Greff et al. | 6,730,113 B2 5/2004 Eckhardt et al. |
| 4,291,701 A 9/1981 Bowman | 6,736,797 B1 5/2004 Larsen et al. |
| 4,306,976 A 12/1981 Bazzato | 6,814,726 B1 11/2004 Lauer |
| 4,338,933 A 7/1982 Bayard et al. | 6,911,025 B2 6/2005 Miyahara |
| 4,346,703 A 8/1982 | 7,083,605 B2 8/2006 Miyahara |
| Dennehey et al. | |
| 4,387,879 A 6/1983 Tauschinski | 7,195,615 B2 3/2007 Tan |
| 4,405,315 A 9/1983 Handt | 7,232,429 B2 6/2007 Moreci |
| 4,412,834 A 11/1983 Kulin et al. | 7,306,197 B2 12/2007 Parrino et al. |
| 4,433,244 A 2/1984 Hogan | 8,469,545 B2 * 6/2013 Sobue |
| | et al. . . . 362/249.02 |
| 4,439,193 A 3/1984 Larkin | |
| 4,475,900 A 10/1984 | |
| Popovich et al. | |
| 4,500,788 A 2/1985 Kulin et al. | |
| 4,503,333 A 3/1985 Kulin et al. | |
| 4,541,829 A 9/1985 Munsch et al. | |
| 4,557,727 A 12/1985 Handt | |
| 4,596,551 A 6/1986 Golinski et al. | |
| 4,617,012 A 10/1986 Vaillcourt | |
| 4,626,245 A 12/1986 Weinstein | |
| 4,626,845 A 12/1986 Ley | |
| 4,655,753 A 4/1987 Bellotti et al. | |
| 4,655,762 A 4/1987 Rogers | |
| 4,675,007 A 6/1987 Terry | |
| 4,695,276 A 9/1987 Shinno et al. | |
| 4,738,668 A 4/1988 Bellotti et al. | |
| 4,755,292 A 7/1988 Merriam | |
| 4,769,017 A 9/1988 Fath et al. | |
| 4,774,415 A 9/1988 Biegel et al. | |
| 4,840,621 A 6/1989 Larkin et al. | |
| 4,869,286 A 9/1989 | |
| Williams et al. | |
| 4,873,446 A 10/1989 | |
| Kreitmair et al. | |
| 4,878,516 A 11/1989 Mathieu. | |
| 4,882,496 A 11/1989 Bellotti et al. | |
| 4,952,812 A 8/1990 Miripol et al. | |
| D310,881 S 9/1990 Larkin et al.. | |
| 5,014,494 A 5/1991 George | |
| 5,047,011 A 9/1991 Caron et al. | |
| 5,057,074 A 10/1991 Suzuki et al | |
| 5,125,911 A 6/1992 | |
| Grabenkort et al.. | |
| 5,184,020 A 2/1993 Hearst et al. | |
| 5,250,041 A 10/1993 Folden et al | |

### Patent Publications

WO 2015/146651
US 2013/0211322

### Non-Patent Literature

https://www.ncbi.nlm.nih/gov/prnc/articles/PMC6478112
https://www.ncbi.nlm.nih/gov/prnc/articles/PMC2722204

1. A totally implantable indwelling peritoneal dialysis catheter that is comprised of several components. Component 1 is called the port and is implanted under subcutaneous tissue of the body at variable sites, depending on the patient's anatomy and surgeon's preference. (Primarily at the abdominal and thoracic region of the body.) Component 2, the implantable catheter. The proximal terminal end of the catheter connects to the port. The proximal and middle portion of the catheter are tunneled under the subcutaneous tissue until it reaches the abdominal wall near the peri-umbilical area. The distal portion of the catheter enters and terminates in the peritoneal cavity. Component 3 is the external perforated sheath with anti-collapsible rib spacers, which is a circumferential covering over the terminating distal catheter.

2. A totally implantable peritoneal dialysis catheter of claim 1, having an injection port (accessible via a hypodermic needle, as one of its components) located at the proximal terminal end of the catheter. (This port is used to infuse a dialysate solution. Additionally, this port is used to evacuate the dialysate solution that is dwelling within the peritoneum for a prescribed length of time.)

3. A totally implantable peritoneal dialysis catheter of claim 1, having an anti-occlusion technology system comprised of anti-collapsible rib spacers for prevention of omental occlusion.

4. A totally implantable peritoneal dialysis catheter of claim 3, containing perforated distal catheter tube.

5. A totally implantable peritoneal dialysis catheter of claim 2, containing an extended surface area (increased diameter) injection access port. (Allows for multiple injections via the hypodermic needle during the life of the device.)

6. A totally implantable peritoneal dialysis catheter of claim 5, containing an internal center pillar support located internally at center port, to prevent the collapsing of the silicone injection diaphragm portion of the port.

7. A totally implantable peritoneal dialysis catheter of claim 1, adjustable in length to accommodate various patient sizes and anatomical variances.

8. A totally implantable peritoneal dialysis catheter of claim 4, containing a rounded anti-clog tip to prevent unintended visceral puncture.

9. A totally implantable peritoneal dialysis catheter of claim 3, contains a perforated external sheath for prevention of omentum occlusion.
